(19) United States
(12) Patent Application Publication
(10) Pub. No.: US 2013/0178716 A1
(43) Pub. Date: Jul. 11, 2013

(54) DEVICE FOR APPLYING A PNEUMATIC PRESSURE STIMULUS IN THE NASAL FOSSAE AND EUSTACHIAN TUBE AT THE TIME OF DEGLUTITION

(71) Applicant: La Diffusion Technique Francaise, Saint-Etienne (FR)

(72) Inventors: Michel MASSARDIER, Saint-Etienne (FR); Gilles CHANTREL, Saint-Etienne (FR); Dominique ESTEVE, Greoux les Bains (FR)

(73) Assignee: La Diffusion Technique Francaise, Saint-Etienne (FR)

(21) Appl. No.: 13/738,214

(22) Filed: Jan. 10, 2013

(30) Foreign Application Priority Data
Jan. 11, 2012 (FR) 1250281

(51) Int. Cl. A61B 5/00

(52) U.S. Cl. CPC A61B 5/4833; A61B 5/4205
USPC 600/300

(57) ABSTRACT

Apparatus for automatically applying a pneumatic overpressure phase, of a value of between 5 and 100 hPa in the Eustachian tube and/or in the nasal fossae of a patient through at least one nostril at the moment of swallowing, includes a gas generator, a gas reservoir, a detection-fluid and control circuit communicating with the gas reservoir and gas generator and leading to a sealed plug in the nostrils forming part of a nosepiece equipped with a control nozzle, a device for measuring at least one characteristic value of a flow of gas during swallowing, a device for automatically comparing the characteristic value with at least one pre-set value, and a device for informing the patient whether the swallowing action is being correctly performed.

### CROSS REFERENCE TO RELATED APPLICATIONS

This application claims priority of French application no. 1250281 filed on Jan. 11, 2012, the entire contents of which is hereby incorporated by reference herein.

### TECHNICAL FIELD

This invention concerns a device for applying a pressure stimulus in the nasal fossae and injecting a dose of air into the Eustachian tube when swallowing, and more particularly, the means for ensuring that deglutition occurs correctly when the pressure stimulus is applied.

### PRIOR ART

The auditory or Eustachian tube is a bone and fibrocartilaginous tube linking the anterior wall of the middle ear to the nasopharynx, i.e. the part of the throat located above the soft palate, posterior to the nose. This auditory tube has several functions: the first is mechanical, in that its closure prevents the introduction of pathogenic agents and nasal secretions into the middle ear. In addition, it exerts mucociliary clearance in its inferior section allowing irritant bodies to be evacuated from the middle ear, whilst its third function of pressure equalization means that the pressure on either side of the ear drum can be equalized to avoid rupture of the latter if there is a major difference in pressure between the external environment and the middle ear.

The Eustachian tube is closed at rest and opens for a fraction of a second during deglutition, in particular. Deglutition involves lowering of the soft palate, retropulsion of the tongue, and elevation of the hyoid bone. During the isthmic phase, i.e. when the food bolus is preparing to pass the oropharyngeal isthmus, velopharyngeal contraction is maximal, the tensor palatini, levator palatini and the pharyngopalatinus muscles contracting in synergy. Protraction of the jaws increases the anterior-posterior diameter of the pharynx and, when it is accompanied by contraction of the oropharyngeal isthmus, causes the tube to open.

It is acknowledged that dysfunction of the Eustachian tube can induce various diseases including recurring infections, repeated otitis, serous or seromucous otitis, or hypoacusis.

In addition, the majority of cases of Eustachian tube dysfunction are associated with primary or secondary defects (Takahasi H, Hayashi M, Sato H, Honjo I. Primary deficits in eustachian tube function in patients with otitis media with effusion. Arch Otolaryngol Head Neck Surg. 1989; 1145; 581-584—Iwano T, Kinoshita E, Doi T, Ushiro K, Kumazawa T. Otitis media with effusion and Eustachian tube dysfunction in adults and children. Acta Otolaryngol Suppl. 1993; 500;66-9) and it is known that the tube, physiologically closed at rest, is opened basically due to the synergistic action of muscles, mainly the tensor palatini muscles.

Physiotherapy exercises have been suggested therefore, to correct Eustachian tube dysfunction, particularly by A. Jacobs, in a thesis entitled La kinésithérapie de la trompe d'Eustache [Physiotherapy of the Eustachian tube] (PhD thesis, Faculty of Medicine, Nancy, 1981, 146 p.).

However, physiotherapy exercises for the Eustachian tube are particularly difficult to perform, especially for children who make up the vast majority of patients with Eustachian tube dysfunction, so that they are rarely prescribed.

A practice is known to help rehabilitate the Eustachian tube in which it is trained to produce a reflex reaction to a pressure stimulus using an appropriate device, thus overcoming the disadvantages of physiotherapy. The Applicant has developed such a device himself, which is in use.

Such a device, which allows a dose of air to be injected into the Eustachian tube when swallowing, i.e. by supplying a defined amount of air, from the appearance of the closure of the soft palate, which lies at the origin of the muscular mechanisms leading to the opening of the Eustachian tube, is described in particular in French Patent FR 2 824 479 owned by the applicant.

This device for applying a pneumatic pressure stimulus in the nasal fossae and in the Eustachian tube when swallowing comprises in particular a pump continuously delivering a flow of air in a supply circuit equipped with a calibrated-leak discharge valve, an air reservoir supplied by a branch circuit of the supply circuit and a control and detection-fluid circuit leading to a nostril plug forming part of a case equipped with a leak jet, the said circuit communicating with the reservoir and being equipped with blocking means with an elastically-deformable membrane.

The device is used by selecting a set overpressure (generally between 15 and 80 mbars), taking a sip of water then closing the mouth and clenching the teeth, respiration then taking place calmly through the nose, then by fitting the nostril plug in an airtight manner. The patient then stops breathing, blocks the air intake and swallows the sip of water while keeping his teeth clenched, the device's air intake only being unblocked after 2 or 3 seconds. The operation is repeated as many times as is necessary, usually about fifteen times.

However, the use of this device is only effective if the swallowing action is performed correctly.

A need therefore exists for a device for re-educating the Eustachian tube by applying a pressure stimulus in the nasopharynx of a patient in which the correct performance of the treatment can be measured.

### Outline of the Invention

One of the aims of the invention is therefore to overcome these drawbacks by proposing an optimised device for re-educating the Eustachian tube by applying a pressure stimulus in a patient's nasopharynx, which is of a simple design, inexpensive and capable of measuring the effectiveness of the treatment.

For this purpose, and according to the invention, a device is proposed automatically to apply a pneumatic overpressure phase, of a value of between 5 and 100 hPa in the Eustachian tube and/or in the nasal fossae of a human being through at least one nostril at the moment of swallowing, the said device comprising at least one gas generator, one gas reservoir and one detection-fluid and control circuit communicating with the gas generator and reservoir and leading to a sealed plug in the nostrils forming part of a nosepiece equipped with a control nozzle; said device is remarkable in that it comprises at least one means of measuring at least one characteristic value of a flow of gas during swallowing, one means of automatically comparing the said characteristic value with at least one pre-set value, and one means of informing the patient capable of indicating whether the swallowing action is being correctly performed.

It is clear that, unlike the devices of the prior art, this device makes it possible to check that the swallowing action is correctly performed in such a way that a number of correctly performed swallowing actions, at a set overpressure, is carried out during the treatment session, making the latter more effective.

According to a first variation, the device comprises at least one means of measuring the variation of the flow and/or the duration of the variation of the flow on swallowing and one means of automatically comparing the value of the variation of the flow and/or the duration of the variation of the flow with at least one pre-set value of variation of flow and/or with at least one pre-set duration of variation of flow.

Alternatively, the device according to the invention comprises at least one means of measuring the overpressure and/or the overpressure duration on swallowing and one means of automatically comparing (30,31) the overpressure and/or the overpressure duration with at least one pre-set value of overpressure and/or at least one pre-set duration of overpressure.

Preferably, the information means indicates the difference between the overpressure value and/or the overpressure duration measured with at least one pre-set overpressure value and/or at least one pre-set overpressure duration.

Furthermore, the said means of automatic comparison compare the overpressure value and/or the duration of measured overpressure with at least one pre-set overpressure value and/or with at least one pre-set duration of overpressure over all or part of at least one of the three overpressure phases applied in the nasopharynx during swallowing, one overpressure increase phase, one so-called overpressure plateau phase during which the overpressure is substantially constant for a set duration, and one decrease phase of the said overpressure.

The pre-set duration of the increase in overpressure is within 0.05 and 0.8 of a second.

The pre-set duration of the overpressure plateau phase is within 0.5 and 5 seconds.

The duration of the decrease phase is within 0.05 and 0.8 of a second.

The pre-set overpressure value consists in a percentage of the overpressure value measured at a pre-set moment of the plateau phase.

Advantageously, the information means comprise visual and/or audible warning means. These visual warning means consist in a screen on which messages can be displayed.

Moreover, the comparison means comprise at least one monitoring means of at least one control means capable of altering the value of the measured overpressure. The said control means preferably consist in an electrovalve.

Advantageously, the device according to the invention comprises means of transferring the measured overpressure values and/or the measured overpressure duration values and/or the pre-set overpressure value and/or the set overpressure duration value and/or of the differences determined by the comparison means.

Preferably, the device comprises a so-called safety valve connected to the flexible pipe in order to limit the pressure in the said flexible pipe to below a set pressure, preferably to below 100 mbars.

### BRIEF DESCRIPTION OF THE FIGURES

Further advantages and features of the invention will emerge from the following description of several variations, given by way of a non-limiting example, of the device for re-educating the Eustachian tube according to the invention with reference to the accompanying drawings, in which:

FIG. 1 is a perspective view of the device for re-educating the Eustachian tube according to the invention,

FIG. 2 is a schematic representation of the device according to the invention,

FIG. 3 is a graphic representation of the overpressure curve measured in the Eustachian tube as a function of time during swallowing,

FIG. 4 is a schematic representation of a variation of the device according to the invention,

FIGS. 5 and 6 are schematic representations of the variation of the device during the different stages of operation of the said device.

### DETAILED DESCRIPTION OF THE INVENTION

There now follows a description of a device for re-educating the Eustachian tube by applying a pneumatic stimulus in the nasal fossae and in the Eustachian tube when swallowing; however, it is clear that the device according to the invention can have other applications such as “Tubomanometry” for example, without departing from the scope of the invention.

In the interests of clarity, in the rest of this description, the same elements have been given the same reference numerals in the different Figures. Moreover, the various cross-sectional views are not necessarily drawn to scale and the sizes of the elements may have been exaggerated in order to make the invention easier to understand.

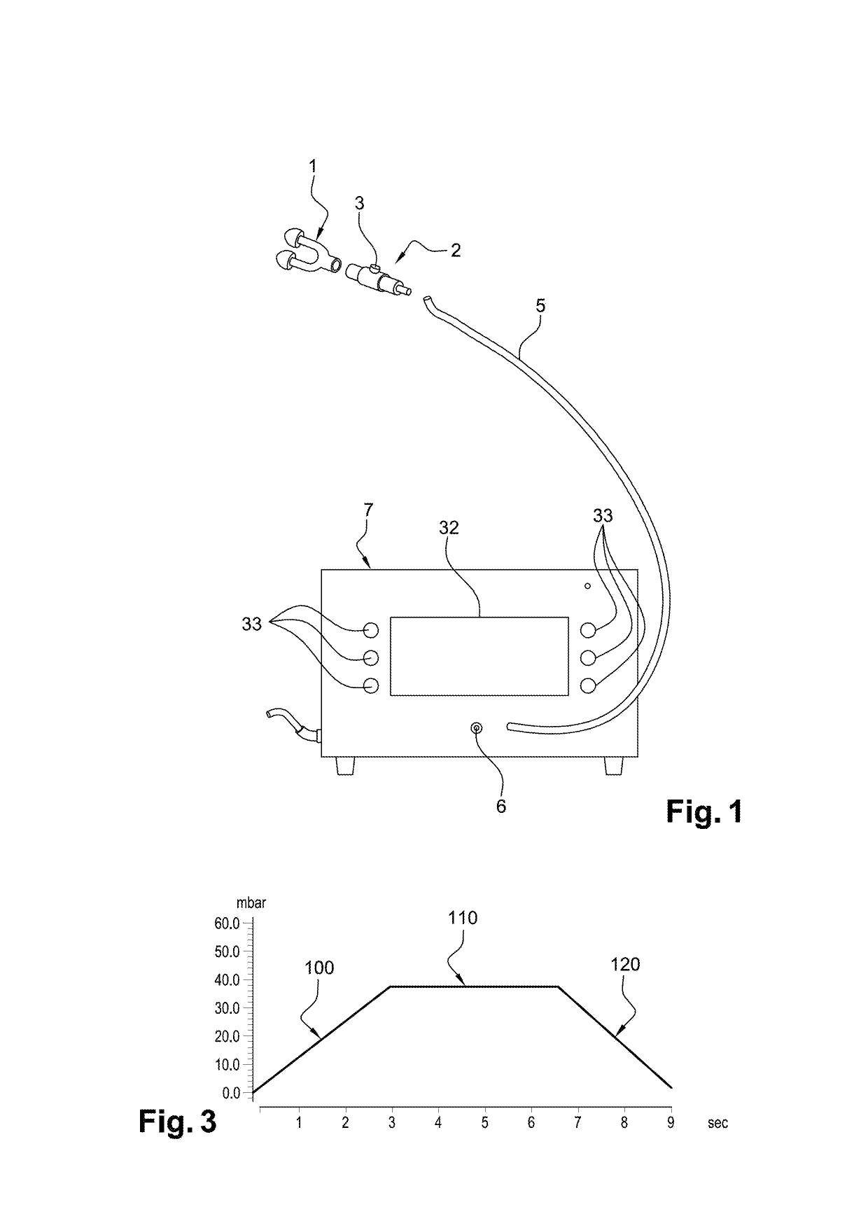

With reference to FIG. 1, the device according to the invention comprises a nostril plug (1) connected to a so-called cylindrical nosepiece (2) comprising a control nozzle (3) and an air inlet nozzle (4) and a flexible pipe connecting the air inlet nozzle (4) to an air outlet nozzle (6) of a case (7).

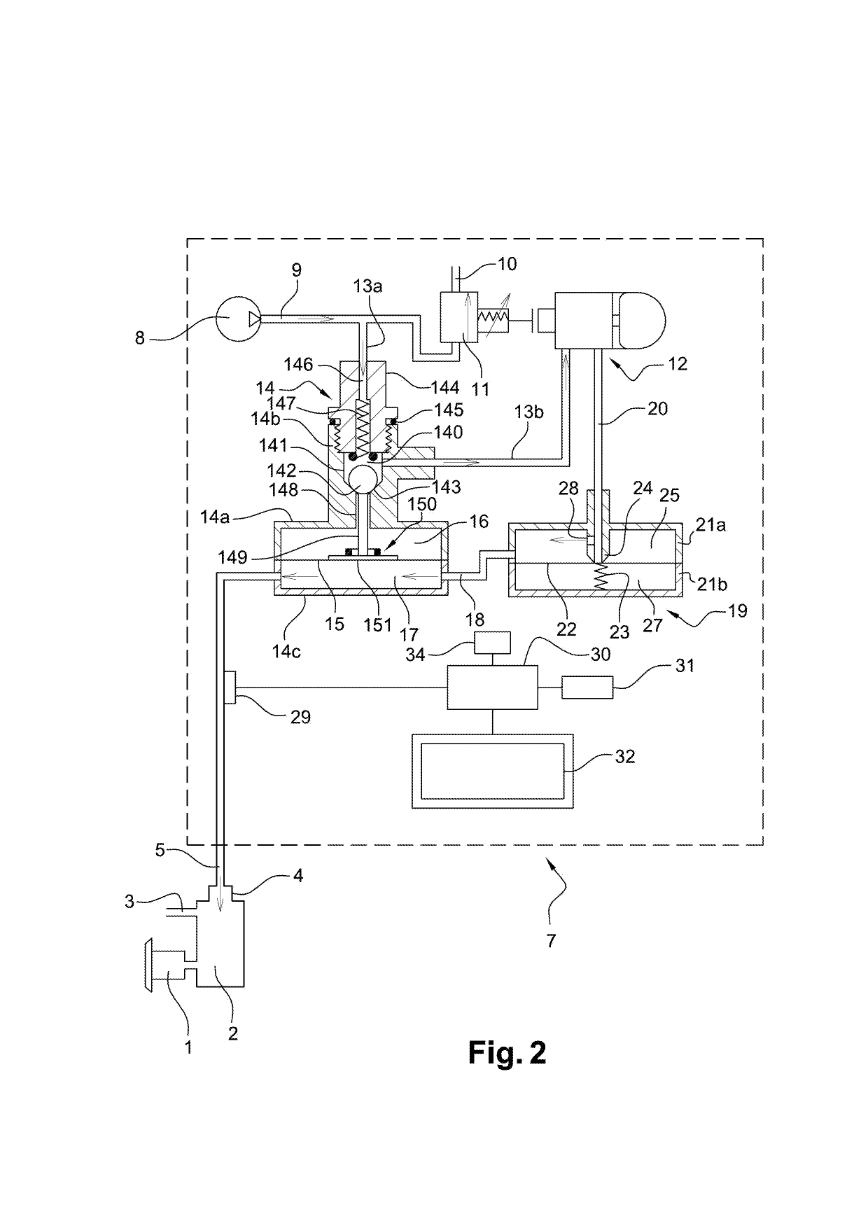

With reference to FIGS. 1 and 2, the case (7) comprises, in the same way as in French patent application FR 2 824 479, a gas generator (8) such as an electric pneumatic pump delivering, into a supply pipe (9), a continuous flow of air under a set pressure determined by a calibrated jet (10) formed by the outlet orifice of an adjustable discharge valve (11). The case (7) also comprises a gas reservoir (12) connected to the supply circuit (9) by an upstream branch (13a) and a downstream branch (13b) of a branch circuit, the branches (13a) and (13b) of the branch circuit being connected to each other by a pneumatic valve (14).

The said pneumatic valve (14) comprises a body in two parts, a first part (14a) in the form of an open box from which a neck (14b) projects and a second part (14c) in the form of an open box. Parts (14a) and (14c) are connected to each other, in an airtight manner, sandwiching between them an elastically deformable membrane (15). This membrane (15) defines, between the two boxes (14a, 14c) a balancing chamber (16) and a control chamber (17). The latter comprises a hole forming the air outlet (6) and is connected by a tube (18) to a swallowing detector (19).

The neck (14b) of the pneumatic valve (14) comprises an axial housing (140) arranged in a metal bush (141) and receiving a metal ball (142), the branch (13b) of the branch circuit leading into the axial housing (140). The bottom end of the axial housing (140) comprises a seat (143) capable of receiving the ball (142) when the latter is in a rest position and the top end of said axial housing (140) is closed by a plug (144) screwed into the neck (14b), compressing an O ring (145) positioned at the top end of the said neck (14b). The said plug (144) comprises an inlet channel (146) communicating with the upstream branch (13a) of the branch circuit and whose bottom part is wider to receive a helical spring (147) which tends to push the ball (142) back into its seat (143). Moreover, the bottom end of the seat (143) has a central bore (148) leading into the balancing chamber (16) and into which extends a rod (149) of a plunger (150) the head (151) of which rests on the membrane (15) of the pneumatic valve (14).

The said swallowing detector (19) is connected to the reservoir (12) by a tube (20) and comprises two parts (21a, 21b) which are assembled together in an airtight manner either side of an elastically deformable membrane (22) which, in the rest position and with the aid of a spring (23), blocks a nozzle (24) connected to the tube (20). This membrane (22) divides the swallowing detector (19) into a control chamber (25) connected to the balancing chamber (16) of the pneumatic valve (14) by a tube (26), and a balancing chamber (27). The nozzle (24) comprises an additional calibrated jet (28) which allows a throughput of air to pass through forming a detection and control flow.

Thus, the nosepiece (2) equipped with a control nozzle (3) forms with the flexible pipe (5), the control chamber (17), the tube (26), the control chamber (25) and the nozzle (24), the detection and control circuit capable of transmitting:
to the nosepiece (2), the detection and control flow delivered by the jet (28);
to the chambers (17) and (25), the above-mentioned increase in flow pressure, after making the ENT environment airtight by swallowing and closing the control nozzle (3);
and to the nostril plug (1), the dose of overpressured air released by the reservoir (12).

Moreover, according to the invention, with reference to FIG. 2, the device comprises a pressure sensor (29) connected to a CPU (Central Processing Unit) (30) including, in a conventional manner, a clock; the said pressure sensor (29) being positioned in the flexible pipe (5) connecting the air inlet nozzle (4) of the nosepiece (2) to the air outlet nozzle (6) of the case (7). This pressure sensor (29) enables the value of the overpressure and/or the duration of the overpressure during the swallowing action to be continuously measured over time, as will be explained later. The CPU (30) is connected to a memory unit (31) and to a display means (32) such as an LCD screen for example.

The memory unit (31) may consist in any memory unit well known to a person skilled in the art such as a DRAM (Dynamic Random Access Memory), a SDRAM (Synchronous Dynamic Random Access Memory), a flash memory or suchlike.

Moreover, it is clear that the central processing unit (30) may not contain an internal clock. In this case, the device according to the invention shall comprise an external clock connected to the CPU (30).

The said CPU (30) also comprises means for comparing the overpressure value and/or the overpressure duration for at least one of the phases to the pre-set overpressure values and/or the pre-set overpressure durations in order to check that the swallowing action is correctly performed during the pressure stimulus, the overpressure curve comprising three phases during swallowing, with reference to FIG. 3, one overpressure increase phase (100), one so-called overpressure plateau phase (110) during which the overpressure is substantially constant for a set duration, and one decrease phase (120) of the said overpressure.

These comparison means consist in a computer programme including algorithms stored either in a CPU (30) or in a memory unit (31). An algorithm is deemed to be a finite set of elementary operations constituting a diagram of calculations or problem solutions.

The said comparison means comprise the first means of comparing the duration of the overpressure increase phase (100) measured by the sensor (29) with a pre-set duration stored in a memory unit. The pre-set duration of the overpressure increase is within 0.05 and 0.8 of a second. Thus, the algorithm forming the first comparison means detects the increase phase (100) of the pressure curve measured as a function of time, then it determines the duration of this increase phase (100) on the basis of the pressure measurement curve. This measured duration of the increase phase (100) is compared with the range of values delimited by two pre-set threshold values recorded in the memory unit (31).

If the measured duration of the increase phase (100) falls outside the range of values delimited by the threshold values, the CPU (30) generates a message indicating a failure of the swallowing action which is then displayed on the screen (32).

If the measured duration of the increase phase (100) falls within the range of values delimited by the threshold values, the CPU (30) may perform a second algorithm corresponding to the second comparison means.

These second comparison means compare the duration of the overpressure plateau phase (110) with a pre-set duration stored in the memory unit (31). The pre-set duration of the overpressure plateau phase (110) falls between 0.5 and 5 seconds. Thus, the algorithm forming the second comparison means detects the plateau phase (110) of the pressure curve measured as a function of time, then it determines the duration of this plateau phase (110) on the basis of the pressure measurement curve. This measured duration of the plateau phase (110) is compared with a value range delimited by the two pre-set threshold values stored in the memory unit (31).

If the measured duration of the plateau phase (110) falls outside the range of values delimited by the threshold values, the CPU (30) generates a message indicating a failure in the swallowing action with is displayed on the screen (32).

By contrast, if the measured duration of the plateau phase (110) falls within the range of values delimited by the threshold values, the CPU (30) may perform a third algorithm corresponding to the third comparison means.

These third comparison means compare the overpressure value measured during the plateau phase (110) with a range of values delimited by the two pre-set overpressure threshold values recorded in the memory unit (31). These two pre-set overpressure threshold values consist respectively in a percentage of the measured overpressure value at a pre-set moment of the plateau phase (110). Thus, the algorithm forming the third comparison means detects the plateau phase (110) of the pressure curve measured as a function of time, then it determines the overpressure value at the start of this plateau phase (110) and the overpressure value at the end of the plateau phase (110). The algorithm then determines the overpressure threshold value as a function of the overpressure value measured at the start of the plateau phase (110) then it compares the value measured at the end of the plateau phase (110) with the previously calculated range of threshold values.

If the overpressure value measured at the end of the plateau phase (110) falls outside the range of values, the CPU (30) generates a message indicating a failure in the swallowing action which is displayed on the screen (32).

Clearly, alternatively, the algorithm can perform any one of the previously described comparison means or any combination of these three comparison means.

Note that the swallowing action failure messages corresponding to the second and third comparison means can also correspond to a loss of seal at the nostril plug (1). In this case, the patient is asked to check that the plug is correctly fitted in the nostrils and the size of plug may be changed. Moreover, the sealed plug may be connected to one nostril only, the other nostril then being blocked.

The device may also comprise audible warning means indicating a failure in the swallowing action, without departing from the scope of the invention.

Note also that the user may in particular set the overpressure value, usually between 5 and 100 hPa, and/or the number of swallowing actions to be performed and/or the overpressure threshold values and/or overpressure durations by means of control and selection buttons (33) located on either side of the screen, shown in FIG. 1.

Advantageously, the device according to the invention comprises means for transferring (34) measured overpressure values and/or measured overpressure duration values and/or the pre-set overpressure value and/or the pre-set overpressure duration value and/or the differences determined by the comparison means. These transfer means consist in a system bus enabling connection to a network and/or in wireless communication means such as Bluetooth®, WI-FI®, Zigbee®, etc., or suchlike.

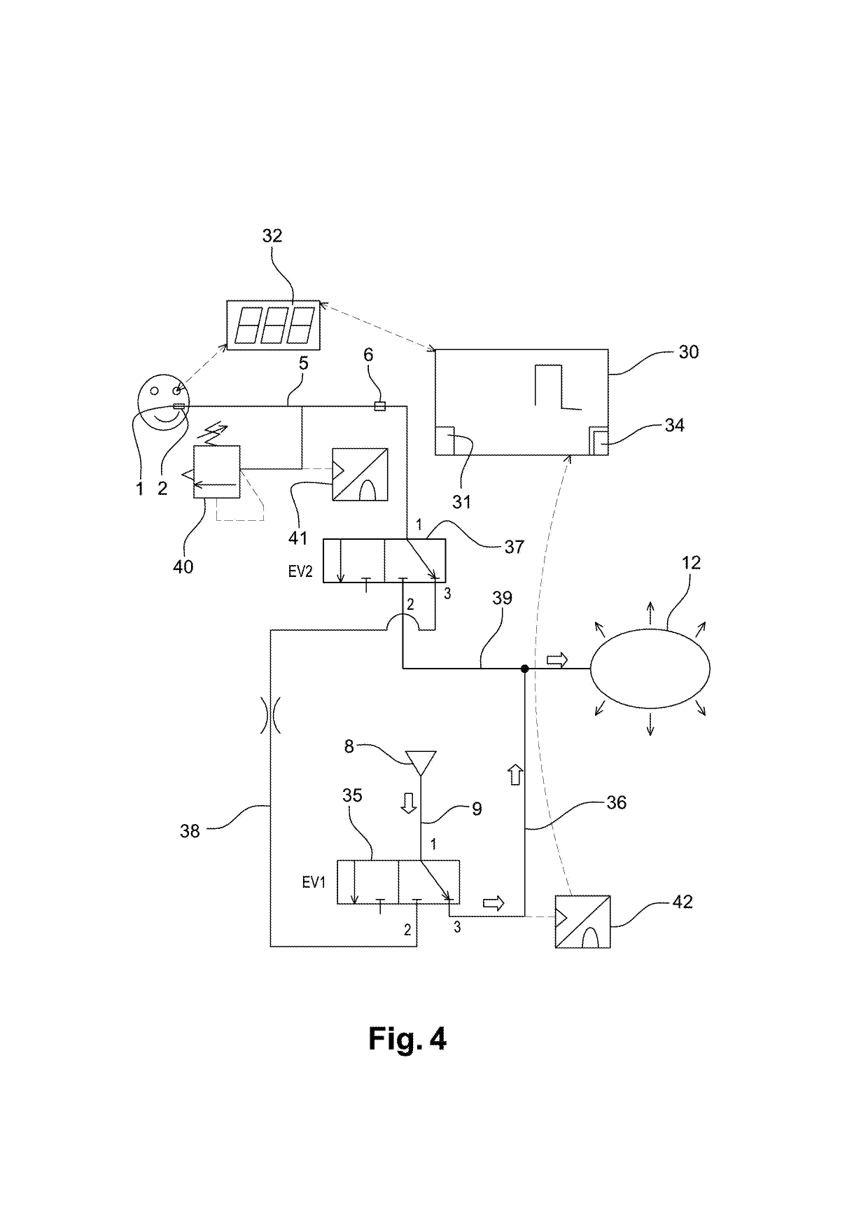

In a variation of the device according to the invention, with reference to FIG. 4, the said device comprises in the same way as before a nostril plug (1) connected to a so-called cylindrical nosepiece (2) comprising a control nozzle (3) and an air inlet nozzle (4) and a flexible pipe (5) connecting the air inlet nozzle (4) to an air outlet nozzle (6) of a case (7), a gas generator (8) such as an electric pneumatic pump delivering, in a supply tube (9), a continuous flow of air under a set pressure. The supply tube (9) is connected to a first three-way electrovalve (35), the first way being connected to the said supply tube (9), the third way being connected to a reservoir (12) by a tube (36) and the second way being connected to a way of a second electrovalve (37) by a tube (38). This second electrovalve (37) also comprises three ways, a first way being connected to a flexible pipe (5) through an air outlet nozzle (6), a third way being connected by the tube (38) to the second way of the first electrovalve (35) and the second way being connected to the reservoir (12) by a tube (39).

The device also comprises a so-called safety valve (40) connected to the flexible pipe (5) so as to limit the pressure in the flexible pipe (5) to below 100 mbars. In fact, when the pressure in the flexible pipe (5) reaches 100 mbars, the valve (40) opens to allow air to escape.

Furthermore, the device according to the invention comprises a first pressure sensor (41) located on the flexible pipe (5) and a second pressure sensor (42) located on the tube (36) supplying the reservoir (12), the said pressure sensors (41) and (42) being connected to a CPU (30) including, in a conventional manner, a clock. The CPU (30) is connected to a memory unit (31) and to display means (32) such as an LCD screen for example. The said CPU (30) also comprises means for comparing the overpressure value and/or the overpressure duration for at least one of the phases to the pre-set overpressure values and/or the pre-set overpressure durations in order to check that the swallowing action is correctly performed during the pressure stimulus.

The device therefore operates in the following way.

With reference to FIG. 4, the first electrovalve (35) is in a so-called rest position, in which the first way is connected to the third way, thus supplying the reservoir (12) which fills up, the second electrovalve (37) also being in a so-called rest position in which the first way connected to the flexible pipe (5) and the third way are connected to the second way of the first electrovalve (35).

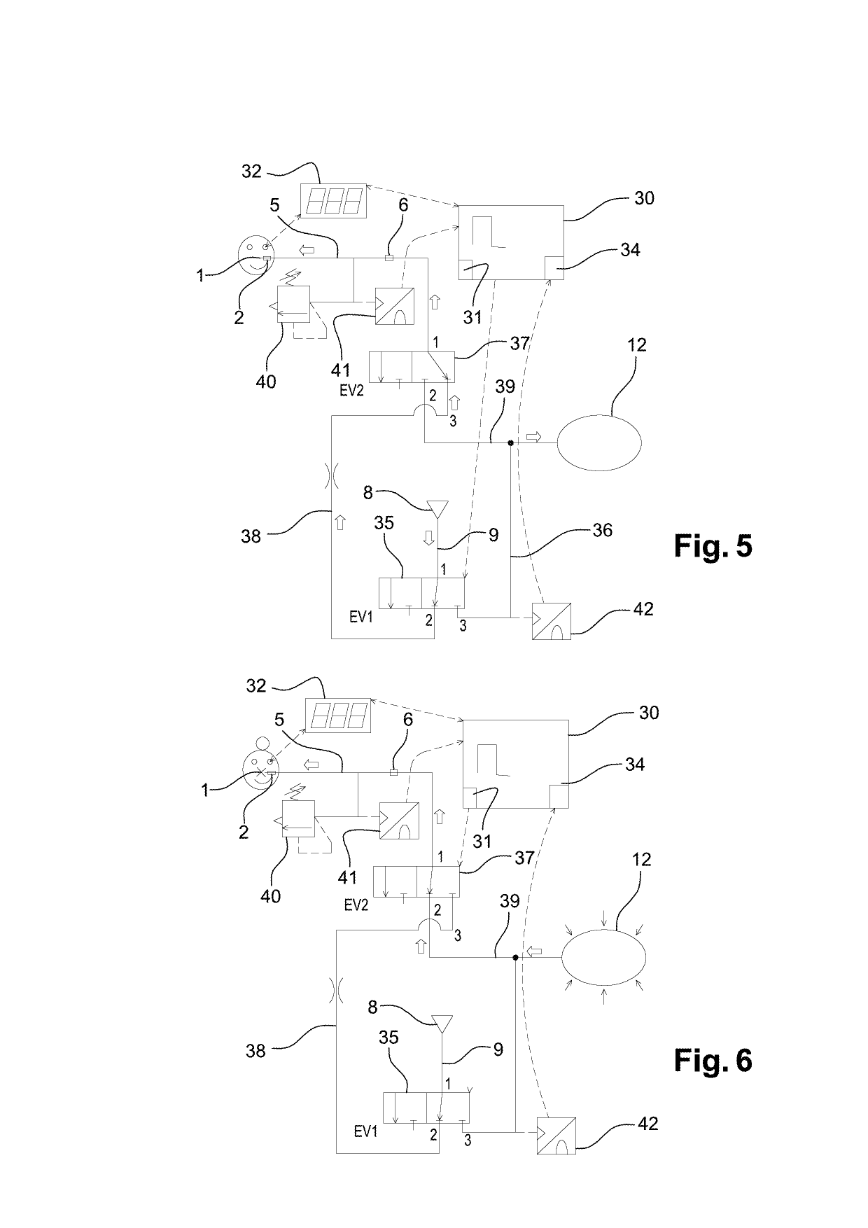

With reference to FIG. 5, when a set pressure is reached in the reservoir (12), the said pressure being measured by the second pressure sensor (42), the CPU (30) activates the first electrovalve (35) so that the first way is now in communication with the second way. In this manner, the reservoir (12) is no longer supplied with air and the pump (8) delivers an airflow, through way 1-3 of the second electrovalve (37), into the patient's nose through the nostril plug (1) connected to the nosepiece (2).

With reference to FIG. 6, when the patient swallows, his soft palate closes and the pressure in the flexible pipe (5) increases. The first pressure sensor (41) continuously measures the pressure in the flexible pipe (5) and when the measured pressure is higher or the same as a pre-set value, the CPU (30) activates the second electrovalve (37) so that the first way is in communication with the second way connected to the reservoir (12) by means of a tube (39). The nostril plug (1) is then supplied with air by the reservoir (12), through way 1-2 of the second electrovalve (37), and the CPU (30) checks the swallowing action as previously described. Once the swallowing action has been completed, the said CPU (30) activates both electrovalves (35) and (37) so that the pump (8) again supplies the reservoir (12) as illustrated in FIG. 4 and a new swallowing cycle can begin.

It goes without saying that, as pressure is dependent on airflow according to Bernoulli's theorem, at least one of the pressure sensors (41, 42) can be replaced by a flow sensor, the CPU (30) then comprising an automatic means of comparing the value of the variation of flow (or overpressure) and/or of the duration of the variation of flow (or overpressure) with at least one pre-set value of variation of flow (or overpressure) and/or at least one pre-set duration of variation of flow (or overpressure), without departing from the scope of the invention.

Lastly, the examples that have just been given are of course merely specific illustrations which are in no way limiting as regards the fields of application of the invention.

1. Device for automatically applying a pneumatic overpressure phase, of a value of between 5 and 100 hPa in a Eustachian tube and/or in a nasal fossae of a patient through at least one nostril at a moment of swallowing, the device comprising a gas generator, a gas reservoir and a detection-fluid and control circuit communicating with the gas reservoir and the gas generator and leading to a sealed plug of a nosepiece equipped with a control nozzle for placement in nostrils; first means for measuring at least one characteristic value of a flow of gas during swallowing, second means for automatically comparing the characteristic value with at least one pre-set value, and third means for informing the patient whether the swallowing action is being correctly performed.

2. Device according to claim 1, wherein the first means comprises means for measuring variation of the flow and/or duration of the variation of the flow on swallowing and the second means comprises means for automatically comparing value of the variation of the flow and/or the duration of the variation of the flow with at least one pre-set value of variation of flow and/or with at least one pre-set duration of variation of flow.

3. Device according to claims 1, wherein the first means comprises means for measuring overpressure and/or overpressure duration on swallowing and the second means comprises means for automatically comparing the overpressure and/or the overpressure duration with at least one pre-set value of overpressure and/or at least one pre-set duration of overpressure.

4. Device according to claim 3, wherein the third means indicates the difference between the overpressure value and/or the overpressure duration measured and at least one pre-set overpressure value and/or at least one pre-set overpressure duration.

5. Device according to claim 3, wherein the second means compares the measured overpressure value and/or the measured duration of overpressure with at least one pre-set overpressure value and/or with at least one pre-set duration of overpressure over all or part of at least one of the following overpressure phases applied in the nasopharynx during swallowing:
overpressure increase phase, overpressure plateau phase during which the overpressure is substantially constant for a set duration, and a decrease phase of the said overpressure.

6. Device according to claim 5, wherein the pre-set duration of the increase in overpressure is within 0.05 and 0.8 of a second.

7. Device according to claim 5, wherein the pre-set duration of the overpressure plateau phase is within 0.5 and 5 seconds.

8. Device according to claim 5, wherein the duration of the decrease phase is within 0.05 and 0.8 of a second.

9. Device according to claim 5, wherein the pre-set overpressure value comprises a percentage of the overpressure value measured at a pre-set moment of the plateau phase (110).

10. Device according to claim 1, wherein the third means comprises visual and/or audible warning means.

11. Device according to claim 10, wherein the visual warning means comprises a screen on which messages are displayed.

12. Device according to claim 3, wherein the second means comprises monitoring means of at least one control means capable of altering the value of the measured overpressure.

13. Device according to claim 12, wherein the control means comprises an electrovalve.

14. Device according to claim 3, further comprising means for transferring measured overpressure values and/or measured overpressure duration values and/or the pre-set overpressure value and/or the set overpressure duration value and/or the differences determined by the second means.

15. Device according to claim 1, further comprising a safety valve connected to a flexible pipe connecting an air inlet nozzle to an air outlet nozzle of a case containing the gas generator in order to limit pressure in the flexible pipe to below a set pressure.
